(19) United States
(12) Patent Application Publication
(10) Pub. No.: US 2010/0267799 A1
(43) Pub. Date: Oct. 21, 2010

(54) Orodispersible pharmaceutical composition of perindopril

(75) Inventors: Patrick Wuthrich, Orleans (FR); Herve Rolland, Olivet (FR); Marc Julien, Silgoy (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Suresnes Cedex (FR)

(21) Appl. No.: 12/803,207

(22) Filed: Jun. 22, 2010

Related U.S. Application Data
(63) Continuation of application No. 10/502,479, filed on Jul. 23, 2004.

(30) Foreign Application Priority Data
Jan. 23, 2002 (FR) 02/00790

(51) Int. Cl. A61K 31/4035; A61P 9/12; A61P 9/00

(52) U.S. Cl. 514/412

(57) ABSTRACT

The invention relates to a solid orodispersible pharmaceutical composition of perindopril, characterised in that it comprises perindopril or a pharmaceutically acceptable salt thereof and granules consisting of co-dried lactose and starch.

The present invention relates to a solid orodispersible pharmaceutical form for the administration of perindopril or pharmaceutically acceptable salts thereof by the oral route, without the simultaneous drinking of a glass of water and without the problem of swallowing.

Perindopril is an antihypertensive compound which especially has an inhibitory action on certain enzymes such as carboxypolypeptidases, enkephalinases or kininase II. It inhibits especially the conversion of the decapeptide angiotensin I to the octapeptide angiotensin II (which is in certain cases responsible for arterial hypertension) by acting on the converting enzyme.

The use in therapeutics of perindopril and pharmaceutically acceptable salts thereof makes it possible to reduce or even to suppress the activity of such enzymes, which are responsible for hypertensive disease or heart failure. The action on kininase II results in an increase in circulating bradykinin and, consequently, in a decrease in arterial tension.

Currently, the tert-butylamine salt of perindopril is administered by the oral route in the form of tablets to be swallowed with half a glass of water. Those perindopril tablets are of use in the treatment of arterial hypertension and congestive heart failure.

The doses of the tert-butylamine salt of perindopril that are currently prescribed range from 1 mg to 8 mg per day, in the form of immediate-release tablets.

Many people, such as children and the elderly, have difficulty in swallowing conventional tablets, the size of which is often not negligible. The problems associated with the ingestion of medicines (choking; “going down the wrong way”; suffocation as a result of obstruction of the throat) are often the cause of poor compliance with dosage regimens or, indeed, of discontinuation of treatment.

The pharmaceutical compositions of the present invention make it possible not only to solve the known problems of a tablet form that has to be swallowed but also to offer a superior medical service which especially allows the quality of life of patients to be improved.

The orodispersible pharmaceutical composition of perindopril has the advantage that elevated plasma levels of active ingredient are obtained rapidly.

The orodispersible pharmaceutical composition according to the invention has the particular characteristic of requiring neither water nor chewing in the course of its administration. It disintegrates very rapidly in the mouth, preferably in less than three minutes and even more preferably in less than one minute.

Many rapid-dissolution forms are described in the prior art. In general, it is common to the previously described technologies that they use a disintegrating agent such as Kollidon® CL (crosslinked polyvinylpyrrolidone), EXPLOTAB® (carboxymethyl starch) and AC DISOL® (crosslinked sodium carboxymethylcellulose).

That disintegrating agent is indispensable to the formulation of the orodispersible tablets and has to be used in conjunction with a direct-compression excipient. The difficulties encountered in the manufacture of such tablets reside in the fact that it is very difficult to obtain tablets having physical characteristics that are constant and reproducible and compatible with the customary handling requirements of tablets.

However, the customarily used mixtures result in tablets of very considerable hardness which is completely unsuitable for rapid disintegration in the oral cavity.

Other orodispersible forms can be produced by using lyophilisation, resulting in very porous solid forms called “oral lyophilisates”. Those forms require the use of a highly specific and complicated industrial process which is lengthy to carry out, yielding a medicament form which has a high cost price. Moreover, the manufacturing process by way of lyophilisation requires a step in which the active ingredient is dissolved in water, which can cause decomposition of the active ingredient.

The present invention enables those problems to be solved. It relates to a solid orodispersible form of perindopril comprising a single excipient of natural origin which allows rapid disintegration and which has a neutral flavour and agreeable texture. The said excipient acts both as binder and as disintegrant. It allows a simple perindopril formulation to be obtained, without using water in the manufacturing process, having excellent suitability for direct compression, resulting in tablets of low friability and of a hardness that is compatible with customary handling methods.

Furthermore, the said excipient allows tablets of very small size to be obtained, which can be given to very young children. It is a requirement for paediatric tablets that administration is made easier, that disintegration in the mouth is very rapid, so as to ensure that the child does not spit the tablet out again, and that the tablets are of sufficient hardness to be handled easily and packaged using simple means (blister pack, suitable unit-dose dispenser).

The orodispersible forms according to the invention make it possible to produce paediatric tablets of very small size (a diameter of 3 mm, a thickness of 1 mm upwards and a weight of 10 mg upwards), which are easy to handle and which disintegrate in the mouth in a few seconds.

More specifically, the invention relates to a solid orodispersible pharmaceutical composition of perindopril, characterised in that it comprises:

perindopril or a pharmaceutically acceptable salt thereof,

and granules consisting of co-dried lactose and starch.

The composition according to the invention may also comprise, for reasons of compound manufacture, one or more lubricants and a flow agent, as well as flavourings, colourings and sweetening agents as conventionally used.

In the pharmaceutical compositions according to the invention, the perindopril is preferably in the form of the tert-butylamine salt.

The invention relates also to the use of granules consisting of co-dried lactose and starch in the manufacture of solid orodispersible pharmaceutical compositions of perindopril.

The term “orodispersible” is understood to refer to solid pharmaceutical compositions which disintegrate in the oral cavity in less than 3 minutes, preferably less than one minute.

The said granules present in the solid pharmaceutical compositions according to the invention correspond to the compositions described in Patent Application EP 00/402159.8. Those granules are characterised by a spherical structure and an advantageous compressibility and are marketed under the name STARLAC®.

The disintegrating properties of the said granules are known for tablets placed in large volumes of stirred liquids. It is especially surprising that, when used in the manufacture of orodispersible forms, the said granules should give especially satisfactory results in terms of disintegration in the mouth, for two reasons.

The first reason is based on the finding that the least water-soluble excipients are the most suitable for the formulation of orodispersible tablets (dissolution, in bringing about an increase in the viscosity of water, slows down its penetration into the tablets) and yet the said, granules contain a large amount of highly water-soluble lactose. Moreover, the starch contained in the said granules is not a “super-disintegrant” agent as used and described in the orodispersible forms of the prior art.

The second is based on the finding that the disintegrant properties of an excipient (used in a tablet), when determined in water using conventional methods, cannot be extrapolated to the behaviour of the same tablet in vivo, in saliva. Disintegration rates in water are measured (in accordance with the European Pharmacopoeia) in an amount of water that is sufficiently large not to reach saturation level in terms of dissolution, whereas in vivo, by virtue of the small volume of saliva, the excipients are at saturation level. Furthermore, the stirring to which the tablets are subjected in the customary test does not reflect disintegration in the mouth. The Applicant accordingly found, during comparative tests, that certain excipients which are known as good disintegrants are not suitable for the preparation of orodispersible forms. Conversely, certain excipients that exhibit average disintegration in water may exhibit advantageous properties in vivo.

The Applicant then found, surprisingly, that the said granules rendered the tablets highly suitable for disintegration in the mouth, that being the case over a wide tablet hardness range, whilst maintaining a low level of friability, which is especially remarkable. Most orodispersible forms of the prior art which disintegrate rapidly in the mouth are highly friable, which is reflected by the need to use a specific packaging and the risk of the tablet disintegrating as soon as it is handled and taken out of its pack.

It is especially remarkable that the above-mentioned criteria of orodispersibility and low friability are maintained over a wide tablet hardness range, that is to say for tablets having a hardness of from 5 to 50 Newtons (preferably from 10 to 20 Newtons).

The pharmaceutical compositions according to the invention are preferably characterised in that they comprise, in relation to the total weight of the tablet:

from 0.1% to 10% by weight of perindopril or a pharmaceutically acceptable salt thereof, preferably from 0.5% to 6%,

from 85% to 99% by weight of STARLAC®.

They may optionally comprise from 0.1% to 3% by weight of lubricating agents such as sodium stearyl fumarate or magnesium stearate (preferably from 0.5% to 1.5%), from 0.1% to 3% by weight of a flow agent such as colloidal silica (preferably from 0.5% to 1.5%) and from 0.1% to 1% by weight of a sweetening agent such as aspartame and/or acesulfame K (preferably from 0.2% to 0.5%).

The following Examples illustrate the invention without limiting it in any way:

Orodispersible Prindopril Tablets

### EXAMPLE 1

Formulation: Finished Tablet of 100 mg

| Constituents | Amount (mg) |
|---|---|
| Perindopril tert-butylamine | 4 |
| Starlac ® | 94 |
| Sodium stearyl fumarate | 1.5 |
| Anhydrous colloidal silica | 0.5 |

### EXAMPLE 2

Formulation: Finished Tablet of 200 mg

| Constituents | Amount (mg) |
|---|---|
| Perindopril tert-butylamine | 8 |
| Starlac ® | 188 |
| Sodium stearyl fumarate | 3 |
| Anhydrous colloidal silica | 1 |

The tablets are prepared by mixing the constituents, followed by direct compression. The hardness of the tablets of Examples 1 and 2 is about 20 Newtons.

In order to determine the disintegration time in the mouth, the orodispersible perindopril tablets described in Examples 1 and 2 were placed in the mouth. In these tests it was found that, for each of the formulations tested, the disintegration time in the mouth was less than 1 minute.

### EXAMPLE 3

Formulation: Finished Tablet of 10 mg

| Constituents | Amount (mg) |
|---|---|
| Perindopril tert-butylamine | 0.0625 |
| Starlac ® | 9.8375 |
| Magnesium stearate MF3 | 0.05 |
| Anhydrous colloidal silica (Aerosil 200) | 0.05 |

Tablets having a hardness of from 5 to 10 Newtons.

### EXAMPLE 4

Formulation: Finished Tablet of 20 mg

| Constituents | Amount (mg) |
|---|---|
| Perindopril tert-butylamine | 0.125 |
| Starlac ® | 19.675 |
| Magnesium stearate MF3 | 0.1 |
| Anhydrous colloidal silica (Aerosil 200) | 0.1 |

Tablets having a hardness of from 10 to 15 Newtons.

### EXAMPLE 5

Formulation: Finished Tablet of 20 mg

| Constituents | Amount (mg) |
|---|---|
| Perindopril tert-butylamine | 0.25 |
| Starlac ® | 19.55 |
| Magnesium stearate MF3 | 0.1 |
| Anhydrous colloidal silica (Aerosil 200) | 0.1 |

Tablets having a hardness of from 10 to 15 Newtons.

### EXAMPLE 6

Formulation: Finished Tablet of 20 mg

| Constituents | Amount (mg) |
|---|---|
| Perindopril tert-butylamine | 1 |
| Starlac ® | 18.8 |
| Magnesium stearate MF3 | 0.1 |
| Anhydrous colloidal silica (Aerosil 200) | 0.1 |

Tablets having a hardness of from 10 to 15 Newtons.

### EXAMPLE 7

Formulation: Finished Tablet of 20 mg (Comprising Sweeteners)

| Constituents | Amount (mg) |
|---|---|
| Perindopril tert-butylamine | 1 |
| Starlac ® | 18.76 |
| Acesulfame K | 0.02 |
| Aspartame | 0.02 |
| Magnesium stearate MF3 | 0.1 |
| Anhydrous colloidal silica (Aerosil 200) | 0.1 |

Tablets having a hardness of from 10 to 15 Newtons.

1. A solid orodispersible pharmaceutical composition comprising:
granules consisting of co-dried lactose and starch, and
perindopril or a pharmaceutically acceptable salt thereof.

2. A composition according to claim 1, wherein the composition disintegrates in the mouth in less than three minutes.

3. A composition according to claim 2, wherein the composition disintegrates in the mouth in less than one minute.

4. A composition according to claim 1, comprising, in relation to the total weight of the composition:
from 85% to 99% by weight of granules consisting of co-dried lactose and starch, and
from 0.1% to 10% by weight of perindopril or a pharmaceutically acceptable salt thereof.

5. A composition according to claim 4, comprising from 0.5% to 6% by weight of perindopril or a pharmaceutically acceptable salt thereof.

6. A composition according to claim 1, further comprising one or more lubricants, a flow agent which is colloidal silica and, optionally, a sweetening agent.

7. A composition according to claim 1, wherein the composition is in the form of a tablet.

8. A tablet according to claim 7, wherein the tablet is obtained by direct compression.

9. A tablet according to claim 8, wherein the tablet has a hardness from 5 to 50 Newtons.

10. A tablet according to claim 9, wherein the tablet has a hardness from 10 to 20 Newtons.

11. A process for the manufacture of solid orodispersible compositions of perindopril, or a pharmaceutically acceptable salt thereof, which disintegrate in the mouth in less than three minutes, wherein the perindopril or a pharmaceutically acceptable salt thereof is mixed with granules consisting of co-dried lactose and starch.

12. A process for the manufacture of solid orodispersible compositions of perindopril, or a pharmaceutically acceptable salt thereof, which disintegrate in is the mouth in less than one minute, wherein the perindopril or a pharmaceutically acceptable salt thereof is mixed with granules consisting of co-dried lactose and starch.

13. A method for treating a living animal body including a human afflicted with a condition selected from arterial hypertension and heart failure comprising the step of administering to the living animal body including a human a composition according to claim 1 which is effective for alleviation of the condition.
